(19) United States
(12) Patent Application Publication
(10) Pub. No.: US 2005/0160669 A1
(43) Pub. Date: Jul. 28, 2005

(54) Method of creating and preserving the identity of non-genetically modified seeds and grains

(75) Inventors: Peter J. Shortridge, St. Paul, MN (US); Kelly Shea Dougherty, Colorado Springs, CO (US); Kate Leavitt Thoresen, Eagan, MN (US); Heather Ninow, St. Paul, MN (US)

Correspondence Address: FISH & RICHARDSON P.C., PO BOX 1022, MINNEAPOLIS, MN, 55440-1022, US

(73) Assignee: Northland Seed & Grain Corp., a Minnesota corporation

(21) Appl. No.: 10/951,289

(22) Filed: Sep. 27, 2004

Related U.S. Application Data
(63) Continuation of application No. 09/251,953, filed on Feb. 17, 1999, now abandoned.

(51) Int. Cl. A01H 5/10

(52) U.S. Cl. 47/58.1 R

(57) ABSTRACT

A method of creating and preserving the identity of non-GMO seeds, grains, and processed food products by selecting seeds which are known to contain non-GMO varieties, planting the non-GMO crop, inspecting grower's operation and machinery to certify that the operation is free of contamination prior to harvest, harvesting the crop, inspecting the processing facility to verify that its operation is free of contamination prior to harvest, tracking all containers holding non-GMO crops each time the crop is moved, and processing the non-GMO crops into containers for shipment where the containers possess tracking information.

### TECHNICAL FIELD

This invention relates in general to a method of creating and preserving the identity of non-genetically modified seeds, grains, and processed product created therefrom, and more particularly to a method of testing and certifying the identity of non-genetically modified seeds and grains at all steps in the planting and growing, processing and packaging, and component and product distribution to preserve the identity of the delivered product, as well as the corresponding product.

### BACKGROUND

It can be seen that there is a need for method of creating and preserving the identity of non-genetically modified seeds and grains. Over the past number of years, genetically modified and genetically engineered seeds and grains are becoming common place within the agriculture industry. The prevalence of these genetically altered products has given rise to a market for non-genetically modified seeds, grains, and processed products created therefrom.

It can also be seen that there is a need for a method to ensure that the non-genetically modified seeds, grains, and processed products are not contaminated during the process of bringing these products to market. The method of creating non-genetically modified seeds, grains, and processed grain products begins prior to the planting of a crop and continues throughout the cultivation and harvest of a crop. Once harvested, the non-genetically modified crop's processing, packaging, and distribution must continue taking steps necessary to prevent contamination of the seeds and grains.

To date, no method of growing, harvesting, processing, packaging, and distributing non-genetically-modified seeds and grains utilizes a comprehensive process which encompasses the entire food production system from farms to supermarkets. Unfortunately, the production of non-genetically modified seeds, grains, and processed products requires such a comprehensive process if the consumer is to have confidence that food products made from non-genetically modified seeds, grains, and processed products have not been contaminated somewhere in the production process.

### SUMMARY OF THE INVENTION

To overcome the limitations in the prior art described above, and to overcome other limitations that will become apparent upon reading and understanding the present specification, the present invention discloses a method of creating and preserving the identity of non-genetically modified seeds, grains, and processed grain products.

The present invention solves the above-described problems by providing a method of creating and preserving the identity of non-GMO seeds, grains, and processed food products by selecting seeds which are known to contain non-GMO varieties, planting the non-GMO crop, inspecting grower's operation and machinery to certify that the operation is free of contamination prior to harvest, harvesting the crop, inspecting the processing facility to verify that its operation is free of contamination prior to harvest, tracking all containers holding non-GMO crops each time the crop is moved, and processing the non-GMO crops into containers for shipment where the containers possess tracking information.

These and various other advantages and features of novelty which characterize the invention are pointed out with particularity in the claims annexed hereto and form a part hereof. However, for a better understanding of the invention, its advantages, and the objects obtained by its use, reference should be made to the drawings which form a further part hereof, and to accompanying descriptive matter, in which there are illustrated and described specific examples of an apparatus in accordance with the invention.

### BRIEF DESCRIPTION OF THE DRAWINGS

Referring now to the drawings in which like reference numbers represent corresponding parts throughout:

FIG. 1 illustrates a process flow chart for the method of creating and preserving the identity of non-genetically modified seeds and grains according to one example embodiment of the present invention for the steps prior to planting through crop harvest.

FIG. 2 illustrates a process flow chart for the method of creating and preserving the identity of non-genetically modified seeds and grains according to one example embodiment of the present invention for the steps from crop harvest through delivery of products to customers.

FIG. 3 illustrates an example application used for the seed certification process according to an embodiment of the present invention.

FIG. 4 illustrates an example DNA test results for the seed certification process according to an embodiment of the present invention.

FIG. 5 illustrates an example chart used for recording sample system per export shipment.

FIG. 6 is an example chart for non-GMO soybean storage chart according to one embodiment of the present invention.

FIG. 7A-F are example inspection chart according to an example embodiment of the present invention.

### DETAILED DESCRIPTION

In the following description of the exemplary embodiment, reference is made to the accompanying drawings which form a part hereof, and in which is shown by way of illustration the specific embodiment in which the invention may be practiced. It is to be understood that other embodiments may be utilized as structural changes may be made without departing from the scope of the present invention.

In general terms, the present invention relates to a method of creating and preserving the identity of non-genetically modified seeds, grains, and corresponding processed products utilizing testing, certification, and documentation of grains, seeds, and processed products throughout the entire growing production and distribution process. These procedures begin prior to planting, proceed through the growing and harvest of grains and seeds, and continue throughout the processing, packaging, and distribution of the grains and seeds.

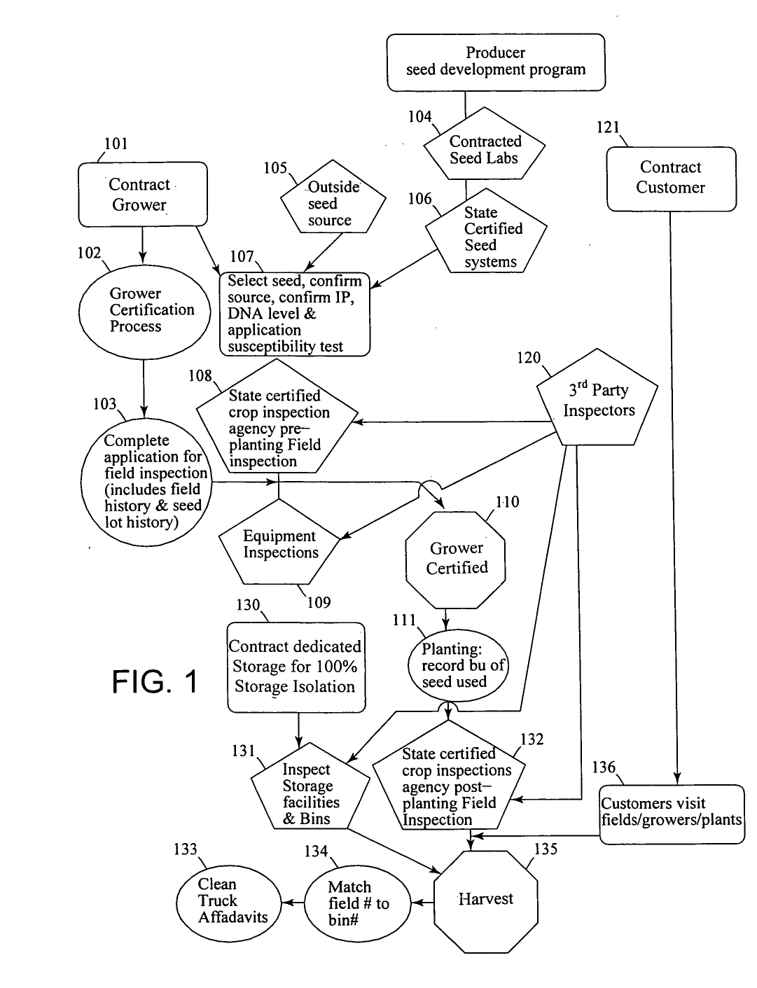

FIG. 1 illustrates a process flow chart for the method of creating and preserving the identity of non-genetically modified seeds, grains, and processed products according to one example embodiment of the present invention for the steps prior to planting through crop harvest. The method begins with step 101 in which a potential grower enters into a contract with a seed and grain producer to plant, grow, and harvest a seed and grain crop according to the methods of the present invention. As part of the process of entering into a contract with the grower, the producer and grower participate in grower education to ensure that both parties understand the goals of the present invention as well as all of the necessary steps that must be followed to conform the crop growing process with the steps of the present invention.

Once under contract, the grower engages in a grower certification and verification process 102. The certification and verification step both ensures satisfactory compliance with the criteria of the present invention regarding the identification as selection of suitable acreage and generates sufficient documentation of compliance with the steps of the present invention necessary to provide customer confidence in the quality of the crop produced. As part of certification and verification step 102, the grower verifies and documents the previous years crop for the identified acreage as well as its neighboring field crop to verify that no problematic isolation and related land issues exist for the identified acreage. These isolation and related land issues include, but are not limited to, plant variety, pollination and cross-pollination, previous crop and volunteer crop, and buffer strips. These issues can be addressed through, but not limited to, inspection, crop and soil tests, buffer strips for the prevention of cross-pollination and contamination, and affidavits. If these corrective steps do not satisfactorily eliminate the issue, the identified acreage is not used to grow a crop according to the present invention.

The grower also completes an application for field inspection 103. This application will include documenting the field history with maps and seed lot history for the past one or more years. The grower will provide complete documentation of seed source, equipment, and storage facilities with maps.

The grower also contracts to use only crop seed which meet the selection criteria of the present invention 107. The selected seed is a Non-GMO variety that is suitable for the land and growing conditions of the identified acreage. The grower, in cooperation with the producer, verify with a seed variety developer that the seed proposed for planting is an Identified Preserve (IP) variety developed from Non-GMO/Non-GEO germ plasm. The IP variety can be verified using plant characteristics such as plant hybrid, maturity, flower color, and leaf shape. In addition, other variety specific characteristics can also be used. The seed developer could be a private company that holds a patent 105, a University Seed Breeding Program 104, Private Laboratory, or similar seed source that utilizes traditional cross breeding to create new seed varieties 106.

The seed selection step 107 also may include a DNA level and application susceptibility test. These tests confirm that the actual supply of the selected seed variety was properly grown and conditioned to protect the integrity of the Non-GMO status. This verification would include inspecting the growers' records to verify where the seeds were obtained; how much seed was purchased; how many pounds per acre the variety requires for planting; how many acres were planted; final yield per acre; and verify that the proper amount of the selected variety of IP seed was purchased and used.

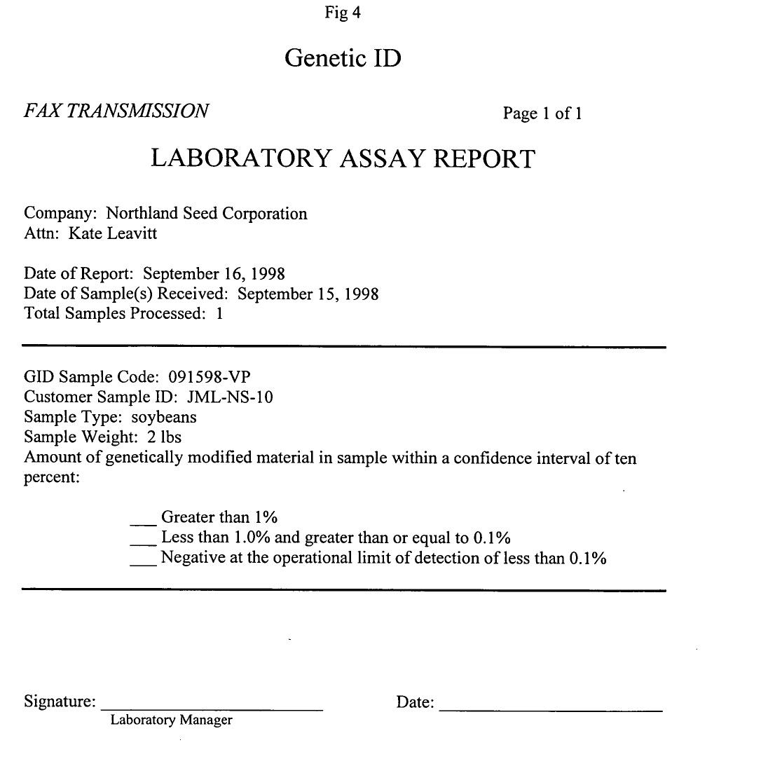

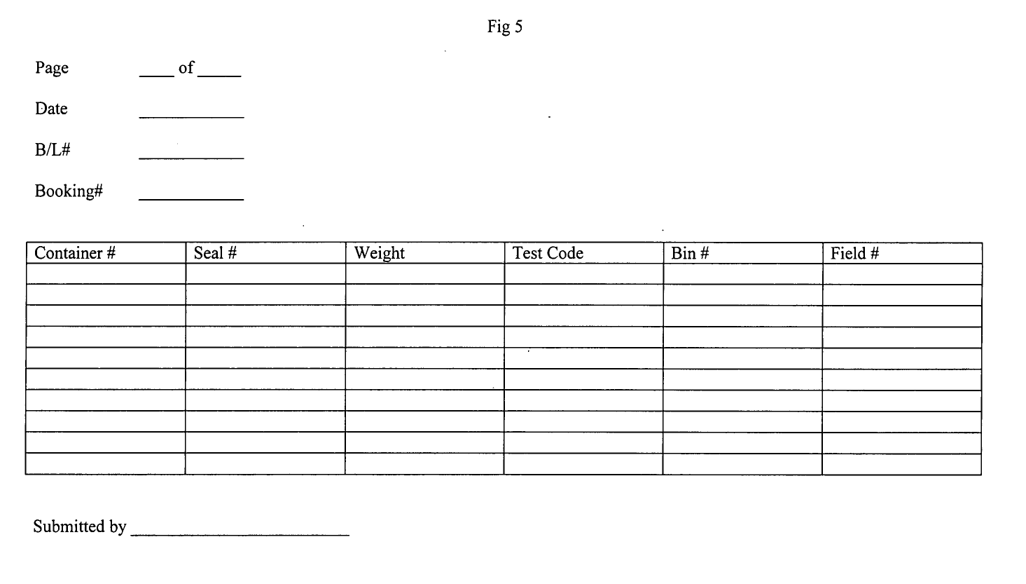

The verification step may also includes additional laboratory testing of the selected seed stock is performed to verify the seed stock is GMO free. These tests include an application susceptibility or detection test and a DNA level test. Satisfactory results from these tests would be influenced by labeling and testing protocols. For example, a two-tiered approach is one possible approach which may be used. For seeds, grains, and processed products which are labeled using language such as “ . . . may contain GMO products,” the DNA level test results in the 1-5% range may be acceptable. For seeds, grains, and processed products labeled as “free of GMO products,” a more stringent standard of 0.01-0.1% results may be required. These more stringent standards may present a technical challenge to testing accuracy of DNA testing laboratories. It is also envisioned that the specific levels may be determined by statutory and/or regulatory authorities for various labels used on the seeds, grains, and process products. Because these levels may vary by country, the testing performed on a particular lot of seeds, grains, and processed products would need to satisfy the requirements for the desired labeling in the country in which the lot is to be sold. FIG. 3 illustrates an example application used for the seed certification process according to an embodiment of the present invention. FIG. 4 illustrates an example DNA test results for the seed certification process according to an embodiment of the present invention.

Once the seed source is selected, the grower certification process may include a pre-planting field inspection 108 and corresponding equipment inspection 109. These inspections are performed by a qualified third-party inspection agency 120. The qualified inspection agency is an independent organization having the required expertise to determine plant purity in regards to varietal purity. The qualified inspection agency would also understand plant specific areas of potential GMO contamination that could occur during the growing season. For example, certain plants may have a greater concern with neighboring field crop variety due to cross-pollination, where other plants may not. A trained inspector would inspect plant characteristics to verify varietal purity. Any problem areas or fields could be rejected prior to harvest. This rejection would prevent contamination of the entire harvest before the crops are stored for shipment and processing. The trained inspector also makes basic yield estimates per field in order to assist in gauging the final quantities to be expected.

According to an alternate embodiment of the present invention, the grower may also contact USDA approved and/or IFOAM approved organic certifiers to inspect and certify the planting, growing, and processing of a crop. Such an activity would permit the grower to grow, and ultimately the producer to produce organic, non-GMO seeds, grains, and processed food products which satisfy all applicable regulatory standards to utilize an “organic” package labeling.

The trained inspector would also verify that all equipment used in the identified acreage is being properly maintained to prevent possible product contamination. These inspections 109 may occur pre-planting and pre-harvest. If the grower owns his or her own equipment, the inspector verifies that it is not used on GMO fields. If the equipment is used on GMO fields, the inspector verifies that proper clean-down steps are taken to prevent chances of contamination. These proper clean-down steps may include, but are not limited to, visual examination, sweeping or blowing out with air, flushing by harvesting a pass and discarding that yield. If the grower uses a contract service for any planting, cultivation, combining, applications, or harvesting activities, the inspector also verifies that these machines have been properly maintained as well. The inspection steps should include inspection of the trucks used to haul harvested product from the fields to storage bins as part of the equipment being inspected.

If a grower passes all of the pre-planting inspection steps, the grower is certified to produce a crop for a given season 110. The certified grower may now plant a crop using selected seeds 107. The grower records the number of bushels of the seeds used as part of planting step 111. During the growing season, the trained inspector 120 will again perform inspections of the fields 132. The inspector will once again verify the grower has taken all of the necessary steps to avoid contamination of the fields. At the appropriate time, the crop is harvested 135.

While the growing process occurs, the producer may enter into contracts with customers for the ultimate delivery of non-GMO seeds and grains 121. Should such a contract be executed, the customer may wish to visit and inspect the fields, growers, and plants used to produce the product 136. During these visits, the inspection results from the pre-planting inspections 108 and the pre-harvest inspections 132 may be viewed as well.

Prior to harvest, additional steps are taken to inspect and approve storage bins and related facilities 131. This process begins with the producer entering into a contract for dedicated storage for the non-GMO product such that 100% storage isolation is maintained. Third party verification prior to usage that the storage bins are dedicated, segregated, contracted, and properly clean and free of GMO product is performed. The harvested crop is tracked with quantities in and out documented using a lot number identification system 134. The lot number identification system tracks lots back to the field where the lot is grown, tracks quantities harvested from a given field, tracks all of the storage locations for a lot from the field to processing, and tracks the date when the lot is moved from a storage bin into a processing plant to prepare for shipment.

The inspection process described above may also be accomplished with a single inspection of the grower in an alternate embodiment of the present invention. According to this alternate embodiment, the grower enters into a contract to grow a desired crop after providing the producer with information which informs the producer that the particular fields being proposed for use by the grower will satisfy the pre-planting criteria (i.e. prior years crop, proper isolation and buffers etc.). When the third party inspector arrives to inspect the fields during the growing season, the inspector verifies all of the information and documentation described above that is checked during the pre-planting inspection 109. The third party inspector also verifies the growers compliance with above-described post-planting/pre-harvest inspections 131 and 132. If either of these inspections reveal unsatisfactory results, the crop from the particular field is rejected.

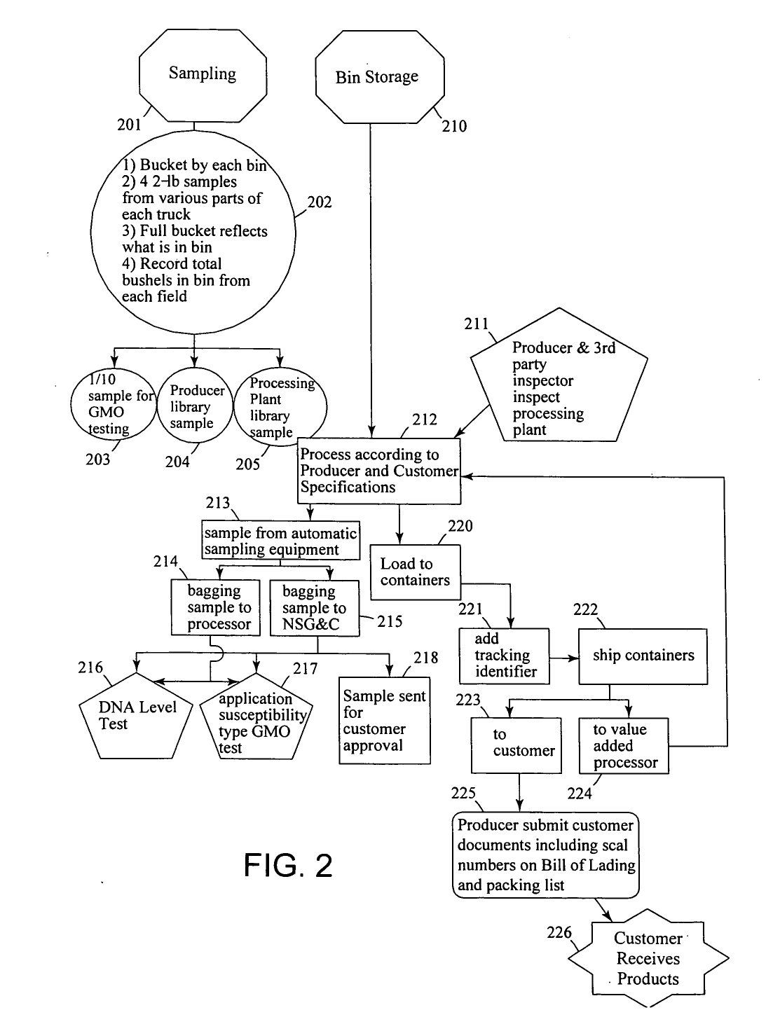

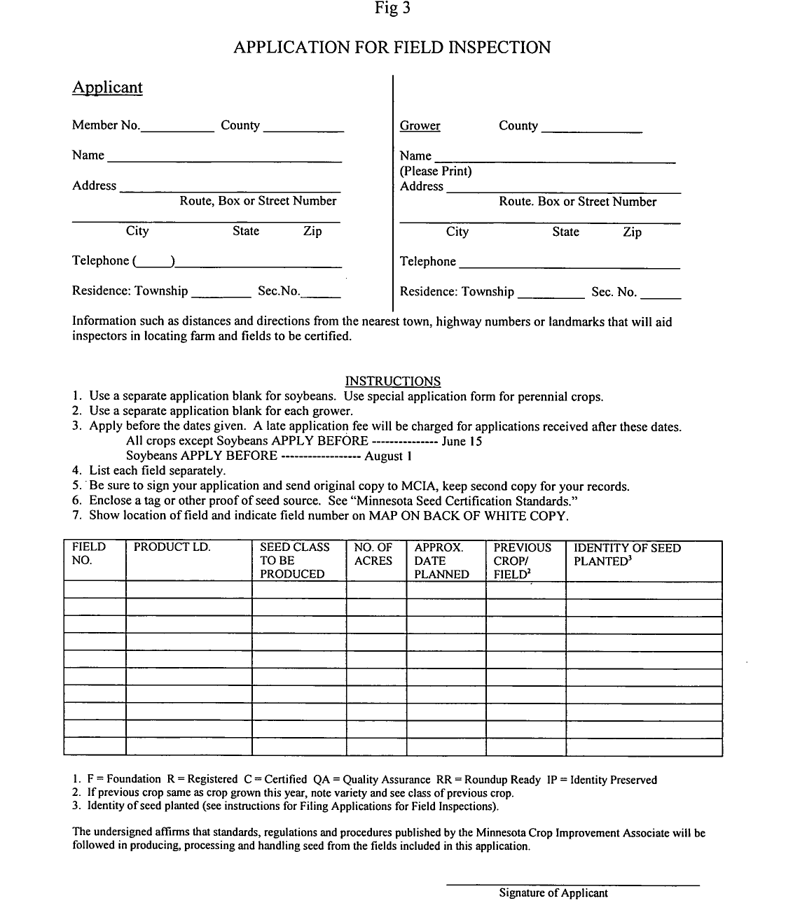

FIG. 2 illustrates a process flow chart for the method of creating and preserving the identity of non-genetically modified seeds, grains, and processed products according to one example embodiment of the present invention for the steps from crop harvest through delivery of products to customers. While the example embodiment presented herein disclosed the process to be utilized by a processing plant that receives a crop that has just been harvested, the process may also be used in all subsequent processing of seeds and grains. For example, non-GMO soybeans may be processed initially to produce clean and bagged soybeans according to the present invention at an initial processing plant. A subsequent plant may process these soybeans into soy meal or soy oil according to the processing disclosed herein without deviating from the spirit and scope of the present invention. In such a case, the product tracking information must be contained throughout the entire processing operation. Additionally, the processing plant must satisfy the cleaning and storage handling requirements discussed herein.

Samples of the product 201 from each truckload from a field should be taken 202 and kept for immediate testing 203 and library sample purposes 204, 205. Clean truck affidavits 133 may be used both from field to storage bin, and from storage to processing plant to verify trucks are properly maintained and proper prevention of contamination has been maintained. In the event that any contamination is found, the corresponding portion of the product is rejected. Each storage bin is numbered and all incoming and outgoing activities records and submitted for inspection.

Additional samples are taken for testing and maintained from bin to processing plant into export-ready ocean container, tote, and bagging. These additional samples are submitted to different laboratories for both application susceptibility tests and DNA level testing. If contamination is found, a second verifying test is performed. If the second test confirms contamination, the product is rejected. Samples are taken and sub-divided into portions for testing, processing plant library sample, producer library sample, and customer review and approval.

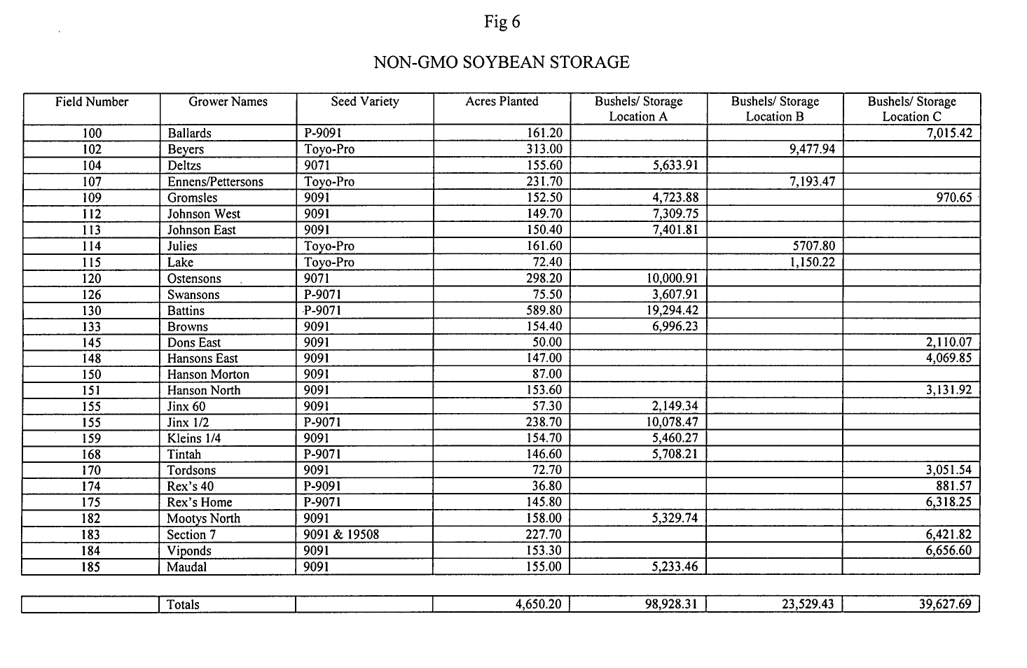

FIG. 5 illustrates an example chart used for recording sample system per export shipment. FIG. 6 is an example chart for non-GMO soybean storage chart according to one embodiment of the present invention.

The harvested and tested crop is taken from bin storage 210 and processed according to customer specifications 212 to generate the deliverable product. Prior to processing of the crop, the processing plant is inspected for compliance with the requirements of the present invention. This inspection is performed by a qualified third party inspection agency such as a state seed and crop inspection agency. This inspection includes, but is not limited, to annual and spot inspections. In general, only processing plants certified by an appropriate industry specific quality designator as having superior quality control should be used. An example of an appropriate industry specific quality designator would be a plant that meets or exceeds the requirements to be a certified seed conditioner. The criteria utilized by this designator would include a set of general requirements, a set of machinery and equipment requirements, and a set of other storage related requirements. All three sets of requirements must be satisfied.

The general requirements begin with copies of all state and federal seed laws and certification requirements must be in the plant operator's possession. The plant operator must be in compliance with all of these laws and certification requirements. Each bag of certified seed must be identified with an identification number for the particular plant. This requirement can also be satisfied if certification tags are sewn into each seed bag. Finally, the plant must be inspected at least once a year to ensure compliance with all laws and regulations.

The machinery and equipment requirements include the use of an air-screen mill equipped with mechanical screen cleaners. The plant should possess on-site weighing and testing equipment necessary to meet its needs. Bag closing equipment should be present on-site if bagged seeds are handled. Compressed air or commercial vacuum cleaning systems must be present to clean equipment, the plant, and all storage containers between the processing of all lots.

The remaining requirements relate to requirements concerning pits, bins, legs, heads and distributors. All elevator pits must be cleanable. It is preferable that all pits possess a metal lining, although the pits must possess a securely fitting cover. Additionally, the valley angle within all pits should be at least 45 degrees.

All bins must be tightly constructed and easily cleanable. Hopper buttons must be made of metal or at least metal lined. All bins must possess a unique identification to permit the tracking of the storage of all seed and grain lots at all points in the storage, processing, and distribution process.

A screen mill must possess at least two legs: one for intake of unprocessed seeds and one for outgoing clean seed. A third leg is, however, recommended for screening only. Elevator boots should be raised at least eight inches above the floor to permit them to be readily opened for cleaning. Plastic cups are optional for elevator legs. All distributors must possess and inspection door to permit easy cleaning. All spouting should be constructed of heavy gauge material and steeply angled. Flex spouting is prohibited. Rivets and bolts should not protrude into the spouts.

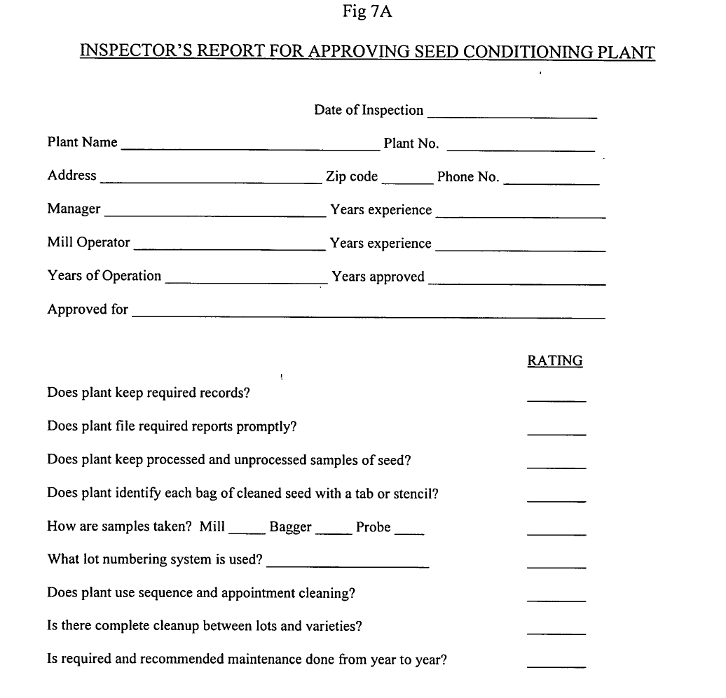

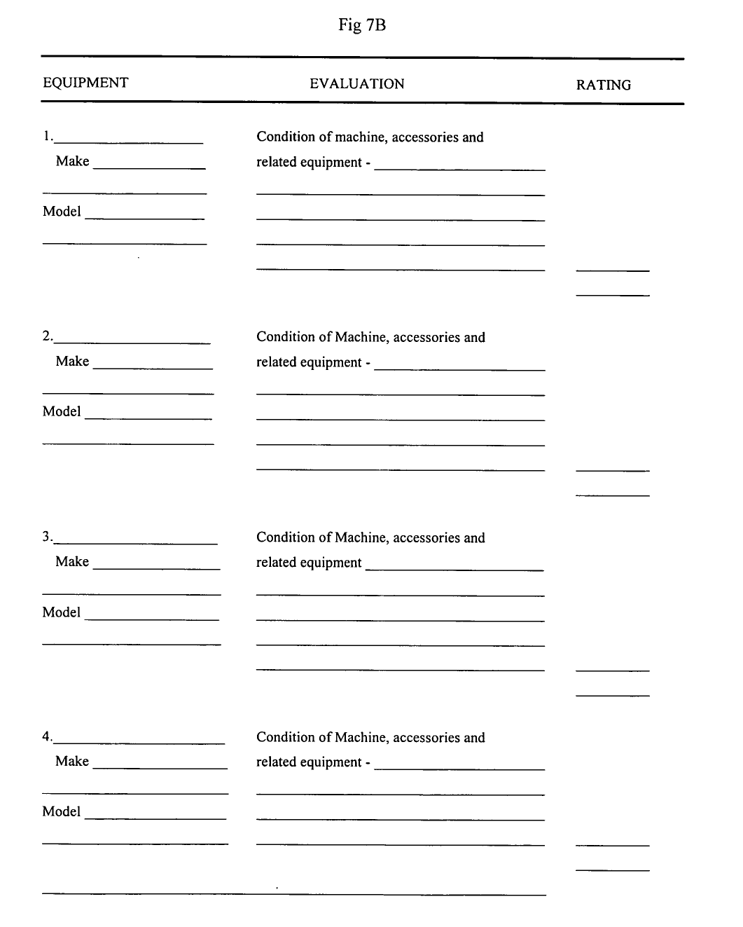

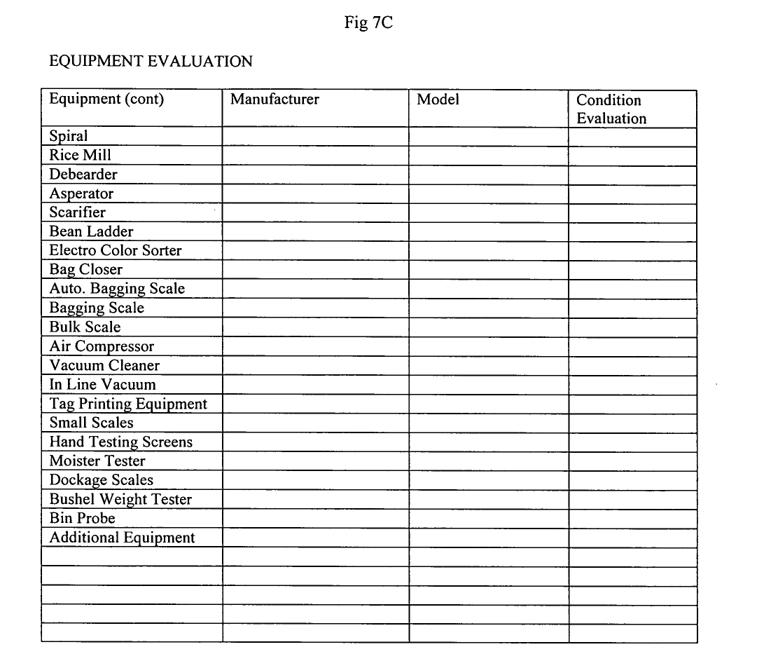

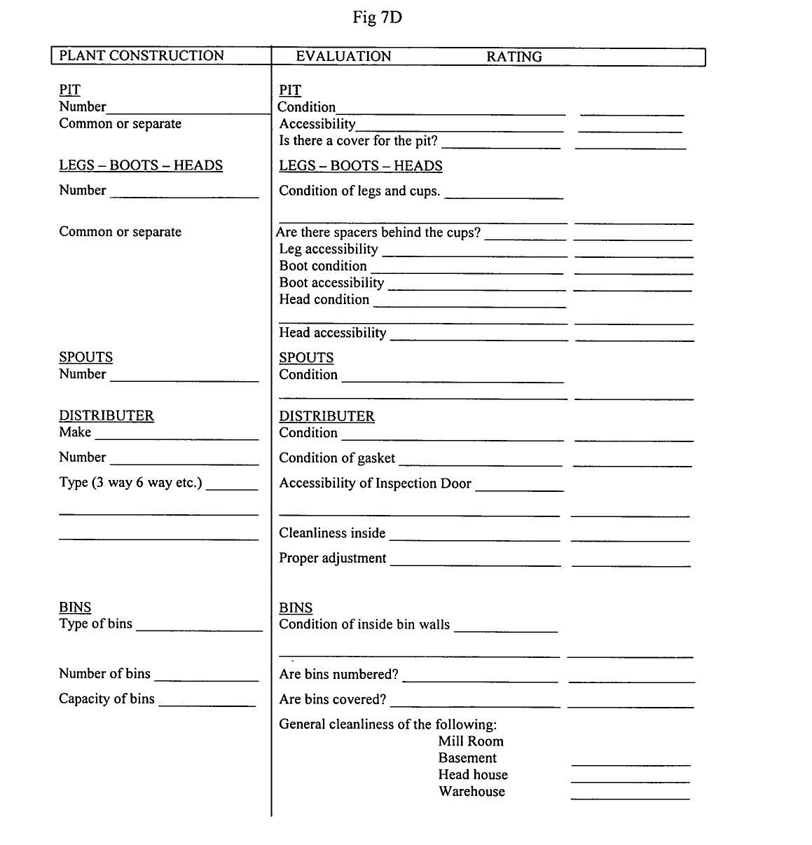

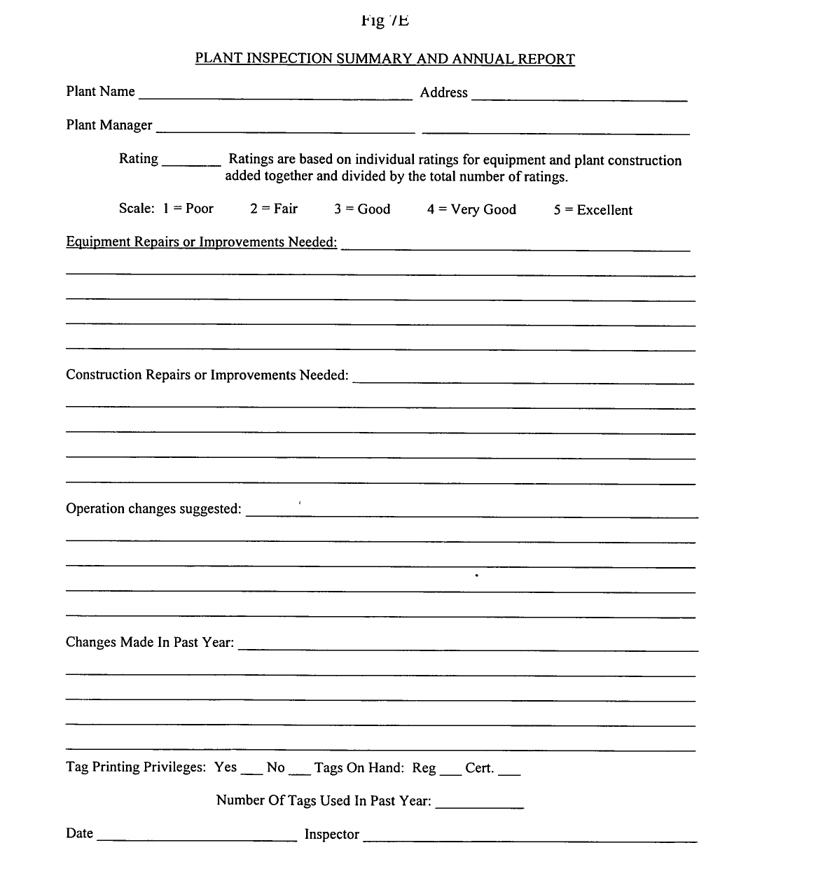

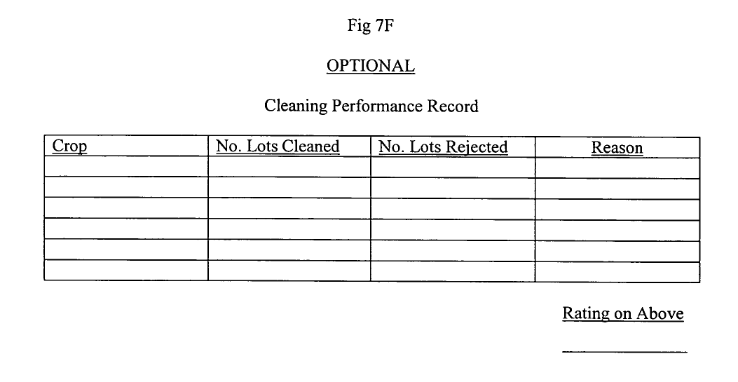

Use of the inspection and quality designator assures the producer and customer that the processing plant possess the ability and understanding of keeping lots and varieties separate, that the processing plant possess the skill of implementing proper clean-down procedures such as a plant, and that the processing plant possess the procedures to properly track lots being processed. With all of these procedures in place, the processing plant would be qualified to process the non-GMO product. Additionally, the processing plant must be capable of maintaining the non-GMO status of the product. To accomplish this task, the processing plant must be state of the art with regards to its equipment, tracking protocol, sampling procedures and the like. The processing plant must be able to clean its machinery and all storage facilities and containers to the highest quality specifications while being able to meet individual customer requirements. FIG. 7A-F are a quality inspection report example inspection report which may be used to verify that a processing plant meets the necessary quality requirements. The above requirements for a processing plant would apply both to processing plants working with bulk grain and to other value adding processing plants such as plants processing soy meal, soy oil crushing facilities, or flour mills.

The processing plant must also have the ability to pack the non-GMO products in several manners, such as bags, totes, bulk to ocean containers, rail cars, or trucks 220. Each package must posses a tracking mechanism to continue the non-GMO lot and tracking procedure used throughout the processing steps. In the case of bulk containers, this procedure may include a seal affixed to the container holding the non-GMO product where the seal referenced the container number. In the case of paper bags or totes, lip-printing, sewn tags or similar manner to affix a lot number system identifier would be attached to the package to facilitate the tracking of the product 221. From this tracking identifier, the field where the product was grown, the variety, storage, and related handling information should all be verifiable. In addition, all packaging must be verified as proper for food-purposes and free of potential to contaminate. From this step in the process, the product is delivered 222 to the value-added processing step 224, or directly to the customer 223, if the product is in its final level of processing for the particular customer in question. The product will ship the product with customer documentation including seal numbers on Bill of Lading and packing list 225. This additional documentation may include, but is not limited to: lot codes, test results, quality certificates, phytosanitary certificates, etc.

As the harvested product is processed within the processing plant, samples are generated using automatic sampling equipment 213. These samples are bagged for further testing by the processor 214 and the producer 215. These samples may be tested for a DNA level test 218 and/or application susceptibility type GMO testing 212. These samples are also maintained within a library 204 in order to permit later testing, should a lot be found to be contaminated at a later date. These samples may also be sent to a customer for approval, as required 218.

The foregoing description of the exemplary embodiment of the invention has been presented for the purposes of illustration and description. It is not intended to be exhaustive or to limit the invention to the precise form disclosed. Many modifications and variations are possible in light of the above teaching. It is intended that the scope of the invention be limited not with this detailed description, but rather by the claims appended hereto.

1. A method of creating and preserving the identity of non-genetically modified seeds, grains, and processed food products the method comprising:
selecting seeds from certified sources which are known to contain only non-genetically modified and non-genetically-engineered varieties;
planting the selected seeds to produce a non-GMO crop;
inspecting a grower operation and machinery to verify that the operation is free of contaminates and conforms to processing and cleanliness criteria prior to harvest;
harvesting the non-GMO crop;
inspecting processing facility to verify that the operation is free of contaminates and conforms to processing and cleanliness criteria prior to harvest;
tracking all containers holding the non-GMO crop each time the crop is moved into and out of a storage container, the tracking includes maintaining the field in which the non-GMO crop was grown, each of the storage containers used to hold the non-GMO crop, and the date of all crop transfers; and
processing the non-GMO crops into containers for shipment, the containers possess tracking information which permits the product to be tracked to the field and containers used to produce the product.

2-23. (canceled)
